(19) United States
(12) Patent Application Publication
(10) Pub. No.: US 2026/0021222 A1
(43) Pub. Date: Jan. 22, 2026

(54) PHOTOPOLYMERIZATION OF NON-MODIFIED PROTEINS

(71) Applicants: Yissum Research Development Company of the Hebrew University of Jerusalem Ltd., Jerusalem (IL); Sheba Impact Ltd., Ramat-Gan (IL)

(72) Inventors: Orit HARARI-STEINBERG, Ra'anana (IL); Shay DUVDEVANI, Ramat Gan (IL); Shlomo MAGDASSI, Jerusalem (IL); Doron KAM, Be'er Sheva (IL); Ayelet BUNIN, Haifa (IL)

(73) Assignees: Yissum Research Development Company of the Hebrew University of Jerusalem Ltd., Jerusalem (IL); Sheba Impact Ltd., Ramat-Gan (IL)

(21) Appl. No.: 19/276,196

(22) Filed: Jul. 22, 2025

Related U.S. Application Data
(60) Provisional application No. 63/673,942, filed on Jul. 22, 2024.

(51) Int. Cl. A61L 27/22; A61L 27/36; A61L 27/38; A61L 27/54; B29C 64/129; B29C 64/30; B29C 71/00; B29L 31/00; B33Y 40/20; B33Y 80/00; C12N 5/077

(52) U.S. Cl. CPC A61L 27/222; A61L 27/3687; A61L 27/3817; A61L 27/3821; A61L 27/54; B29C 64/129; B29C 64/30; B29C 71/0009; B33Y 40/20; B33Y 80/00; C12N 5/0655; A61L 2430/02; A61L 2430/06; B29K 2089/00; B29L 2031/753; C12N 2513/00; C12N 2533/54

(57) ABSTRACT

A biological 3D printed multilayered scaffold is provided, which comprises a crosslinked native or non-modified protein containing a di-tyrosine matrix: the scaffold being configured for containing living cells introduced thereto during printing or post-printing.

### TECHNOLOGICAL FIELD

The invention generally contemplates 3D printing of multilayered protein-based objects and products made therefrom.

### BACKGROUND

3D bioprinting holds great promise in the field of personalized medicine, including the fabrication of medical implants. There are two main approaches to fabricating biological implants: printing a bio-scaffold followed by cell-seeding or printing compositions containing living cells (cell-laden). The most common 3D bioprinting technology is based on Direct Ink Writing (DIW), in which the object is formed by extrusion. This technology is widely used due to the relatively low cost of printers and the case of the printing process, while the ink could be made, stored, and deposited inside a syringe. However, this technology has several challenges and limitations. First, the resolution of the printed object is typically low since it is often dictated by the extrusion nozzle diameter, which is a few millimeters. Second, if the printing composition contains cells, they experience shear forces that negatively affect their viability. Third, a rapid fixation mechanism is required, often based on rheology or printing within a reactive liquid, thus limiting the printing compositions to a small range of polymers.

In Digital Light Processing (DLP), 3D objects are constructed through a layer-by-layer polymerization process. Optimizing printing parameters, such as light intensity and duration, as well as controlling the printing head movement, is essential for obtaining high-quality 3D objects. Most bioinks used in DLP consist of one or a combination of two main components: synthetic monomers with functional groups capable of undergoing polymerization, such as acrylamide or poly (ethylene glycol) diacrylate; and biopolymers with covalently bonded reactive groups, such as gelatin-methacrylate (GelMA).

Protein-based scaffolds printed by stereolithography processes offer advantages over the commonly used materials. Biocompatible scaffolds are temporary templates that provide a suitable microenvironment for cells to adhere, proliferate, and differentiate, ultimately facilitating the regeneration of damaged or diseased tissues.

Cartilage tissue engineering has emerged as a promising approach to address the limited regenerative capacity of cartilage tissue. Two main therapeutic strategies have been extensively explored: in vivo and ex vivolin vitro cartilage tissue engineering. In vivo approaches involve implanting engineered cartilage constructs directly into the defect site within the living body, allowing the construct to benefit from the native environment and vascular supply, potentially promoting more biomimetic cartilage formation and integration with the surrounding tissue. Conversely, ex vivo or in vitro approaches involve culturing and maturing the engineered cartilage construct in a controlled laboratory environment before implantation, enabling precise control over culture conditions and monitoring of construct properties. Implementation of both approaches requires a biocompatible scaffold that will be populated by the cells and support their differentiation and maturation into mature tissue. Thus far, efforts have focused on developing advanced scaffolds that can support and guide cartilage regeneration. Scaffolds with tailored architecture, mechanical properties, and bioactive cues are developed in an effort to provide a biomimetic environment for chondrocyte growth, proliferation, and cartilage matrix production. Additionally, the advent of 3D bioprinting technology has enabled the fabrication of highly precise and customized scaffolds, offering the potential for patient-specific implants and improved integration with native cartilage.

### GENERAL DESCRIPTION

Non-functionalized gelatin, an abundant protein derived directly from collagen, plays a crucial role in forming the Extra-Cellular Matrix (ECM). Gelatin possesses many advantages, including good biocompatibility, solubility, degradability, easy acquisition, low price (˜0.7$ per gram, compared to ˜300$ per gram of GelMA), and lower antigenicity compared to collagen. Moreover, gelatin maintains the arginine-glycine-aspartic acid (RGD) peptide sequence, which promotes cell adhesion, proliferation, and differentiation, and therefore can play a crucial role in fabricating efficient bio-scaffolds for medical implants.

In recent years, gelatin emerged as a promising platform for cartilage tissue engineering since it closely mimics the extracellular matrix (ECM) of native cartilage tissue, providing a familiar microenvironment for chondrocytes. The gelatin-containing scaffolds can be engineered with controlled pore structures and mechanical properties to support cell adhesion, proliferation, and differentiation. Recent advancements in 3D printing and microfluidic technologies have enabled the fabrication of highly organized porous gelatin scaffolds with uniform pore sizes, further enhancing their potential for cartilage regeneration. As with other water-based printing compositions, there are noteworthy challenges in printing gelatin, including gelling at high concentrations and low temperatures, and the necessity for water-soluble constituents only. Among them, the need for water-soluble photo-initiators (PIs) poses a significant obstacle due to limited availability and low efficiency.

Photopolymerization is frequently initiated by ultraviolet (UV) or visible light; however, UV light may increase DNA mutations and chromosomal instability, which can further lead to apoptosis and affect cell viability. Thus, visible light may be more biocompatible than UV light for bioprinting. As demonstrated herein, use of a water-soluble PI, such as [Ru(bpy)₃]²⁺, in the presence of sodium persulfate (SPS) (referred to herein as a Ru/SPS system) enables crosslinking of native gelatin when irradiated with light within the visible spectrum (400-700 nm), having an absorbance peak at 450 nm. Upon irradiation, the Ru(II) complex and persulfate composition undergo photolysis, forming Ru(III) and sulfate radicals. The Ru(III) species functions as an oxidizing agent, which facilitates the oxidation of the tyrosine residues present to generate tyrosine radical intermediates. These tyrosine intermediates then undergo arene coupling with neighboring tyrosine groups, to generate a di-tyrosine, a process further stabilized by the persulfate radicals and involves removal of a hydrogen atom. In addition, although the Ruthenium complex is considered expensive, it requires a significantly lower concentration (as low as 0.06 wt. %) compared to a commonly used efficient water-soluble PI, such as LAP (˜0.5-1 wt. %). When normalizing the price per concentration in the formulation, it is about ˜10 times cheaper. Therefore, the herein suggested formulation significantly reduces the cost of protein-based formulations (considering the low price of gelatin and the price per formulation of the Ruthenium complex).

The technology disclosed herein generally concerns visible light-induced 3D printed protein multilayers suitable for use as scaffolds for inducing or stimulating growth of tissues in a living subject. As disclosed herein, the visible light-induced 3D printing involves printing of stacked layers of a native or non-modified protein having an abundance of tyrosine moieties, such proteins including gelatin, collagen and others, in presence of a water-soluble photo-initiator (typically a Type-II photo-initiator capable of inducing photopolymerization under visible light), and optionally further in presence of cells. The generation of the multilayered biological scaffold (e.g., as an implant or cartilage), by way of printing, as disclosed herein, does not involve the use of synthetic monomers or polymerizable materials of synthetic or semisynthetic origins.

Thus, in some cases and typically, products of the invention are free of synthetic or semisynthetic monomers, oligomers or other synthetic or semisynthetic polymerizable materials.

As used herein, scaffolds and products of the invention are referred to as “biological”. The technology enables manufacturing of objects and scaffolds that essentially or mainly consist the non-modified proteins, and other biological components of non-synthetic origins/sources. Other structural elements are not present in the objects and scaffolds, and as such they are fully biodegradable and safe. While synthetic or semisynthetic or non-biological materials may be used in the manufacturing of scaffolds and other products of the invention, such materials are not typically present in the final manufactured scaffold or product in any substantial amount. The term thus encompasses a product of the invention that is substantially free (containing for example, up to 5 wt % of such materials) of synthetic, semi-synthetic or non-biological material. In some cases, the amount of such materials may be up to or not exceeding 4, 3, 2, 1, 0.5, 0.1, 0.01 wt %.

In a first aspect, there is provided a biological 3D printed multilayered scaffold (to be used as an implant or cartilage), the scaffold comprising a crosslinked native or non-modified protein structure comprising di-tyrosine bonds (forming a di-tyrosine matrix or a structure); the scaffold being configured to contain or hold living cells and maintain the cells in a viable state over a period of time.

As disclosed herein, the cells may be introduced into the scaffold during printing or post-printing. In other words, the cells may be introduced as part of the formulation that is printed, as disclosed herein, or into the manufactured scaffold prior to instilling the scaffold in the body, or after it has been instilled. Also, as further noted herein, the cells may be cells added to the scaffold from a different source, not autologous cells, or cells which migrate from the site of instillation into the bear, cell-free scaffold.

The 3D scaffolds of the invention are wholly or fully and completely formed by printing, thus referred to as “printed” objects. Typically, the printing method is vat photopolymerization. While, in some embodiments, the scaffold may be used in combination with another structural material or other additives, the 3D printing may consist printing by vat photopolymerization of all elements, sections, and parts of the scaffold itself.

The invention further contemplates a biological 3D printed multilayered scaffold, the scaffold comprising a crosslinked native or non-modified protein comprising di-tyrosine bonds (matrix); the scaffold comprising cells introduced thereto during printing or post-printing, wherein the scaffold is for inducing or stimulating cells or tissue growth or generation in vivo.

Further provided is a biological 3D printed multilayered scaffold for inducing or stimulating tissue growth in vivo, the scaffold comprising printed layers of a crosslinked native or non-modified protein comprising di-tyrosine assembly; the scaffold comprising cells capable of inducing or stimulating the growth in vivo.

The scaffold of the invention is a biological structure, a structured implant or a structured cartilage that is configured as an in vivo implant for supporting or delivering cells carried thereby to the site where the implant is positioned in a body (human or animal). The scaffold structure may be of any 3-dimentional size and shape and may be designed as a generic scaffold or may have a size or a shape dictated by the site of instillation or implantation. Scaffolds of the invention are generally multilayered structures comprising a matrix of di-tyrosine moieties, and arranged into two or more printed layers of the non-modified protein, formed by utilizing visible light-mediated printing methods to generate scaffold containing cells or scaffolds that are structured to receive cells prior to instillation in a subject's body. Unlike approaches relying on synthetic materials or chemically modified biopolymers, the technology used for preparing scaffolds of the invention harnesses the inherent properties of the protein, such as gelatin, to create the biocompatible scaffold structures.

The protein-based scaffolds, comprising crosslinked proteins as disclosed, typically solely comprise the crosslinked proteins. Where cells are present, the amount or concentration of the cells may vary. The amount of the protein in a formulation used for forming the scaffold may vary, as it was found to be printable at the maximum documented concentration of 30 wt. %. Such high concentrations of the protein allow fabrication of overhanging objects and open embedded tubes or hollow structures with a compressive modulus akin to soft tissues. Thus, scaffolds of the invention may be intricate structures with mechanical properties ranging between soft and hard.

The scaffold may be prepared in two general forms: a form containing cells (herein referred to as cell-laden scaffolds) and a form that is capable of receiving the cells after the scaffold has been prepared (herein referred to as a cell-seeding scaffold). Cell adhesion and growth onto and within the protein-based scaffolds were evaluated by examining the two forms. The biological experiments indicated that both the cell-seeding and cell-laden forms enabled making 3D cultures of cells such as chondrocytes within the protein scaffolds. This underscores the potential of utilizing non-modified protein-only bioinks in light-mediated printing to produce intricate 3D objects with high fidelity, paving the way for advancements in regenerative tissue engineering.

The protein used in scaffolds of the invention is a native non-modified protein having an abundance of tyrosine amino acids. In other words, the protein is a protein purified from its natural source, e.g., blood, serum, plasma, etc., and which structure and function is preserved. The protein is not a modified protein, a recombinant protein or any other protein not substantially preserving its characteristics as in the host tissue or organism. It should be noted that while some of the characteristics of the protein, when in its natural environment, may be lost due to isolation, the isolation process does not introduce or use any such steps that substantially alter the attributes of the protein.

The protein may be characterized as being rich with or having an abundance of tyrosine amino acid moieties. Such a protein may be, in some embodiments, gelatin, resilin, collagen, albumin, silk fibroin and mixtures thereof.

Typically, the non-modified protein does not comprise di-tyrosine moieties, or comprises less than an effective number of di-tyrosine moieties. The di-tyrosine moieties are formed during printing according to methods of the invention.

In some embodiments, the protein is gelatin or a mixture of proteins comprising gelatin.

The invention further provides a biological 3D printed multilayered scaffold comprising a layered crosslinked native or non-modified gelatin structure comprising di-tyrosine matrix; the scaffold comprising cells introduced thereto during preparation, e.g., vat photopolymerization, or post-preparation, e.g., post-printing.

The invention further contemplates a biological 3D printed multilayered scaffold, the scaffold comprising a layered crosslinked native or non-modified gelatin having di-tyrosine functionalities; the scaffold comprising cells introduced thereto during printing or post-printing, wherein the scaffold is for inducing or stimulating cells or tissue growth or generation in vivo.

Further provided is a biological 3D printed scaffold for inducing or stimulating tissue growth in vivo, the scaffold comprising a crosslinked native or non-modified gelatin comprising di-tyrosine functionalities; the scaffold comprising cells capable of inducing or stimulating the growth in vivo.

The cells contained in the scaffold may be selected based on their ability to induce or stimulate tissue growth in vivo. Generally speaking, as the site of instillation of a scaffold of the invention is not limited, the cells capable of stimulation of tissue growth may vary. In some embodiments, the cells are selected to induce or stimulate growth or generation of cartilaginous or bone tissues. The cells may be selected from chondrocytes, osteoblasts, osteocytes, osteoclasts and others.

In addition to the cells, various minerals and factors may also be added to the scaffold. Factors that may be incorporated into the scaffold for cartilage and bone tissue engineering include, without limitation:

### Growth Factors:

TGF-β (Transforming Growth Factor Beta) family: TGF-β1, TGF-β2, TGF-β3, BMPs (Bone Morphogenetic Proteins): BMP-2, BMP-4, BMP-6, BMP-7, IGF-1 (Insulin-like Growth Factor 1),
FGF (Fibroblast Growth Factor) family: FGF-2 (basic FGF), FGF-18,
PDGF (Platelet-Derived Growth Factor),

### Differentiation Factors:

Sox9 (for chondrogenic differentiation),
Runx2 (for osteogenic differentiation),
Osterix (for osteogenic differentiation),
Wnt signaling molecules (for both chondrogenic and osteogenic pathways),
Indian Hedgehog (IHH) (for chondrocyte differentiation),

### Additional Factors:

VEGF (Vascular Endothelial Growth Factor): for vascularization in bone tissue,
PTHrP (Parathyroid Hormone-related Protein): for regulating chondrocyte differentiation,
GDF-5 (Growth and Differentiation Factor 5): for joint development,
   Factors/Molecules that Promote Cell Adhesion, Proliferation, and Survival:
Fibronectin: Promotes cell adhesion and is involved in wound healing processes,
Laminin: Essential for cell adhesion and tissue reorganization,
Collagen: Supports cell adhesion and provides structure to tissues,
EGF (Epidermal Growth Factor): Stimulates proliferation of epithelial cells,
VEGF (Vascular Endothelial Growth Factor): Promotes formation of new blood vessels and supports endothelial cell survival,
PDGF (Platelet-Derived Growth Factor): Encourages cell division and is involved in wound healing,
IGF-1 (Insulin-like Growth Factor 1): Promotes proliferation and survival of cells in various tissues,
FGF-2 (Fibroblast Growth Factor 2): Supports proliferation and differentiation of many cell types,
Vitronectin: Promotes cell adhesion and is involved in blood coagulation processes,
Integrins: Cell surface receptors that mediate adhesion to extracellular matrix proteins,
Cadherins: Cell-cell adhesion molecules essential for maintaining tissue structure,
Heparin Sulfate: Regulates growth factor activity and promotes cell adhesion,
Hyaluronan: Supports tissue hydration and promotes cell migration,
Osteopontin: Promotes cell adhesion and is involved in bone mineralization processes,
Tenascin: Involved in wound healing processes and embryonic development.

Scaffolds of the invention may comprise one or more of the factors and growth factors disclosed herein.

The 3D digital light-mediated printing methods may be or may involve deposition of a protein formulation in a layer-by-layer sequence, whereby each layer is irradiated and cured before the next layer is deposited; or in a formulation bath that is illuminated or irradiated to cause curing of the formulation (namely, polymerizing the tyrosine units to generate a matrix having d-tyrosine bonds). The light-mediated curing may involve stereolithography, DLP or volumetric printing, where a light beam is scanned over the formulation bath to form a layer of hardened material. A platform may be used to move the structure being formed downwards by one layer thickness as the scaffold is formed. In other configurations, the light curing may be similarly used, with the light beam focusing on the bottom of the bath, or at a specifically selected location within the bath to construct the scaffold structure. In some embodiments, the printing method is or may include Digital Light Processing (DLP), stereolithography (SLA), Direct Ink Write (with light irradiation), Polyjet printing, volumetric printing, two-photon polymerization printing, extrusion deposition and others.

In some embodiments, the light curing method or the 3D printing method is vat photopolymerization, e.g., utilizing light to selectively cure (harden) a liquid photopolymer resin in a vat (container), building objects layer by layer.

A method of preparing a scaffold of the invention may thus comprise visible light-mediated 3D printing of a plurality of stacked layers of a protein-based formulation to form a 3D multilayered scaffold structure, wherein the protein-based formulation comprises or essentially consists at least one native non-modified protein having an abundance of tyrosine amino acids, a water-soluble photo-initiator (typically a Type-II photo-initiator capable of inducing photopolymerization under visible light), at least one electron acceptor, a liquid carrier (e.g., water or an aqueous medium) and optionally living cells.

As used herein, the term “essentially consisting” refers to a composition of the formulation which at least 98% (e.g., by weight) thereof is formed of the components noted. For example, the expression “ . . . essentially consists at least one native non-modified protein having an abundance of tyrosine amino acids, a water-soluble photo-initiator (typically a Type-I photo-initiator capable of inducing photopolymerization under visible light), at least one electron acceptor, a liquid carrier (e.g., water or an aqueous medium) and optionally living cells” should be understood to mean that at least 98% (e.g., by weight) of the formulation is formed of the materials mentioned. In some embodiments, the term encompasses at least 98%, at least 98.5%, at least 99%, at least 99.5%, or 100% (typically by weight).

The light-mediated printing method is carried out under conditions that generate the di-tyrosine functionalities or matrix and which ensure survivability or viability of the protein and cells which may optionally be incorporated in the scaffold, during printing. These conditions include carrying out the process at a pH, an ionic strength and generally in presence of additives that do not cause damage to the protein, or lysis or any other breakdown of the cells. The conditions also involve maintaining the protein-based formulation or the formulation bath (depending on the method used) at a temperature not exceeding about 37° C. when cells are used, and not exceeding about 50° C. when cells are not present.

The invention further provides a method for preparing a multilayered biological scaffold, the method comprising irradiating by a light having a wavelength in the visible range an aqueous formulation comprising at least one native non-modified protein having an abundance of tyrosine amino acids, a water-soluble photo-initiator (typically a Type-II photo-initiator capable of inducing photopolymerization under visible light), at least one electron acceptor and optionally living cells, causing the tyrosine amino acids to form di-tyrosine structures transforming said formulation into said biological scaffold.

Further provided is a method for preparing a biological scaffold, the method comprising layer-by-layer irradiating by a light having a wavelength in the visible range an aqueous formulation comprising at least one native non-modified protein having an abundance of tyrosine amino acids, a water-soluble photo-initiator compositions (capable of inducing photopolymerization under visible light), and optionally living cells, causing the tyrosine amino acids to form di-tyrosine structures transforming said formulation into said biological scaffold.

In some embodiments, the formulation further comprises cells, as disclosed herein.

In some embodiments, the formulation is free of cells. In some embodiments, following production of the scaffold, cells are seeded onto the scaffold; alternatively, the cell-less scaffold may be treated with a formulation of cells to cause said cells to seed or penetrate the scaffold or associate to a surface or a surface region of the scaffold.

In some embodiments, the aqueous formulation comprises at least one native non-modified protein, such as gelatin, a water-soluble photo-initiator (typically a Type-II photo-initiator capable of inducing photopolymerization under visible light), at least one electron acceptor and optionally living cells, as disclosed herein.

In some embodiments, the formulation comprises gelatin and cells selected from such capable of inducing or stimulating growth of cartilaginous tissue or bone.

The number of cells in a formulation of the invention may be between 1×10⁶ to 1×10⁸.

The water-soluble photo-initiator is typically capable of inducing photopolymerization under visible light. The photo-initiator may be based on combining a ruthenium-based photo-initiator and a water-soluble electron acceptor. Such a photo-initiator may be a material containing the ion [Ru(bpy)₃]²⁺.

The amount of the photo-initiator in the formulation may range between 0.5 and 20 mM. The ratio of photo-initiator to the electron acceptor can be in the range of 1:2 up to 1:20. In some embodiments, the ratio photo-initiator:electron acceptor can be between 1:2 and 1:20, such as 1:2, 1:3, 1:4, 1:5, 1:6, 1:7, 1:8, 1:9 . . . 1:15, 1:16, 1:17, 1:18, 1:19 or 1:20. Any intermediate ratio is also included.

In some embodiments, the photo-initiator is provided with an electron acceptor such as sodium persulfate (SPS). In some embodiments, the photo-initiator is a ruthenium-based material and the electron acceptor is SPS. In other words, in some embodiments, non-modified gelatin containing the amino acid tyrosine may be caused to undergo a photopolymerization reaction to form di-tyrosine bonds that enable crosslinking of the protein in solution, in the presence of [Ru(bpy)₃]²⁺-based photo-initiator and sodium persulfate (SPS).

The formulation may comprise one or more additional additives. The additives may be selected from active ingredients and non-active ingredients, such as photo-absorber for resolution improvement (e.g., phenol-red), or organic monomers and water-soluble Type 1 or Type 2 photo-initiators, and additional structural components (such as polysaccharides) for subsequent crosslinking. The scaffold may for example comprise active agents that improve the condition of the subject, stimulate healing of an injured tissue or organ, facilitate hastened healing, reduce pain, etc. Such agents may include analgesic agents, anesthetic agents, anti-infective agents, antibiotics, antiviral agents, anti-inflammatory agents, peptide drugs, steroids, etc.

Scaffolds of the invention, being non-inflammatory, non-immunogenic, and which can be prepared having the appropriate properties, may be used as an effective implantable soft- or hard-tissue scaffold material. The scaffold may be incorporated into a tissue of a human or an animal subject, by injection or implantation, to achieve biocompatibility without inducing a pathophysiological response. In some embodiments, the scaffold is provided in order to be durably retained in the tissue. Alternatively, the scaffold may be transiently retained in the subject, and is provided as substantially biodegradable.

The invention further provides a method for delivering a scaffold into a subject's body, e.g., by injecting or implanting said scaffold into a tissue, the scaffold comprising at least one protein, e.g., gelatin, and cells, wherein the protein and cells are selected to achieve one of more of (i) recruitment of host cell infiltration, (ii) promotion of tissue growth, and (iii) tissue regeneration in a subject.

Further provided is a method for inducing or stimulating cells or tissue growth or generation in vivo or ex vivo, the method comprising instilling or positioning a biological 3D printed scaffold according to any aspect of the invention in a tissue or an organ, wherein said scaffold comprises cells capable of recruiting of host cell infiltration, promotion of tissue growth, and/or tissue regeneration.

In some embodiments, the inducing or stimulating cells or tissue growth or generation comprises any one or more of promoting cell adhesion, promoting tissue reorganization, promoting or stimulates proliferation of cells such as epithelial cells, promoting formation of new blood vessels, promoting or supporting endothelial cell survival, promoting cell division, promoting survival of cells, regulating factor activity, supporting or promoting tissue hydration, and promoting embryonic development.

In some embodiments, the scaffold is a cell-laden scaffold. In some embodiments, the scaffold is a cell-seeding scaffold.

In some embodiments, the scaffold is prepared by irradiating with a light having a wavelength in the visible range an aqueous formulation comprising at least one native non-modified protein having an abundance of tyrosine amino acids, a water-soluble photo-initiator compositions (capable of inducing photopolymerization under visible light), and optionally living cells, causing the tyrosine amino acids to form di-tyrosine structures transforming said formulation into said scaffold.

In some embodiments, the formulation comprises living cells, as disclosed herein.

In some embodiments, the formulation is free of cells. In some embodiments, following instillation of the scaffold, the scaffold is treated with a formulation comprising living cells, and the method further comprises causing said cells to seed or penetrate the scaffold or associate to the scaffold surface.

In some embodiments, the scaffold comprises or is formed of a non-modified gelatin. In some embodiments, the scaffold is a gelatin scaffold comprising cells selected from such capable of inducing or stimulating growth of cartilaginous tissue or bone. In some embodiments, the scaffold comprises living cells, the number of which may be between 1×10⁶ to 1×10⁸ or between 1×10⁶ to 1.5×10⁶.

In some embodiments, the instilled scaffold may further comprise active agents that improve the condition of the subject, stimulate healing of an injured tissue or organ, facilitate hastened healing, reduce pain, etc. Such agents may include analgesic agents, anesthetic agents, anti-infective agents, antibiotics, antiviral agents, anti-inflammatory agents, peptide drugs, steroids, etc.

In some embodiments, the scaffold is instilled or positioned or incorporated into a tissue of a human or an animal subject, by injection or implantation, to achieve biocompatibility without inducing a pathophysiological response.

The invention further provides a tissue, in vivo or ex vivo, instilled with or associated to a scaffold of the invention.

In some embodiments, the tissue exhibits an improvement as compared to a tissue control not instilled with the scaffold and tested in the same way (for example, as mentioned herein). The improvement may be any one or more of improved or increased cell adhesion, improved or increased tissue reorganization, improved or increased proliferation of cells such as epithelial cells, improved or increased formation of new blood vessels, improved or increased endothelial cell survival, improved or increased cell division, improved or increased survival of cells, improved or increased factor activity, improved or increased tissue hydration, and improved or increased embryonic development.

In some embodiments, the improvement is determined by a method such as viability tests (e.g., cell counting, luminescence-based assays), histology, Immunostaining and others.

The invention further provides a 3D printed multilayered biological scaffold, the scaffold comprising a crosslinked native or non-modified protein comprising di-tyrosine bonds. This may be regarded a cell-less scaffold.

In some cases, a scaffold according to the invention contains living cells introduced thereto during printing or post-printing.

In some cases, the scaffold contains materials that enhance cells viability and proliferation; and/or further comprising factors for cartilage and bond tissue engineering.

In some cases, the factors are as disclosed herein, e.g., are growth factors, differentiation factors, and/or factors promoting adhesion, proliferation or survival.

In some cases, the scaffold contains living cells and materials that enhance cells viability and proliferation; and/or further comprising factors for cartilage and bond tissue engineering.

In some cases, where the scaffold contains living cells, the factors are growth factors, differentiation factors, and/or factors promoting adhesion, proliferation or survival.

In some cases, the scaffold is configured for inducing or stimulating tissue growth in vivo, the scaffold comprising printed layers of a crosslinked native or non-modified protein comprising di-tyrosine bonds; and cells capable of inducing or stimulating the growth in vivo.

In some cases, the protein is selected from gelatin, collagen, albumin, silk fibroin and mixtures thereof.

In some cases, the scaffold is formed by vat photopolymerization.

In some cases, where living cells are present, the living cells are selected from chondrocytes, osteoblasts, osteocytes, osteoclasts and mixtures thereof.

The invention also provides a 3D printed multilayered gelatin scaffold, the scaffold comprising a crosslinked native or non-modified gelatin having a di-tyrosine bonds; the scaffold comprising living cells introduced thereto during printing or post-printing.

In some cases, the scaffold of the invention consists biological materials and, as stated herein, is free of synthetic or semisynthetic materials. Such synthetic or semisynthetic materials, which may be used in the manufacturing of the scaffold, are not substantially present in the manufactured scaffold.

The invention further provides a method of preparing a scaffold according to the invention. The scaffold may be a cell-less scaffold or one containing cells. Thus, the method may comprise visible light-mediated 3D printing of a protein-based formulation to form a 3D multilayered scaffold structure, wherein the protein-based formulation comprises or essentially consists at least one native non-modified protein having an abundance of tyrosine amino acids, a water-soluble photo-initiator, at least one electron acceptor, a liquid carrier and optionally living cells.

In some cases, the formulation used in the manufacturing method is maintained at a temperature not exceeding about 37° C. when cells are used, and not exceeding about 50° C. when cells are not present.

In some cases, a cell-less scaffold is immersed in a formulation comprising the cells; or a cell-less scaffold is treated with a formulation of cells to cause said cells to seed or penetrate the scaffold or associate to the scaffold surface; or a cell-less scaffold is configured to receive thereinto cells migrated subsequent to instillation in the tissue.

In some cases, the protein is gelatin and the formulation comprises cells capable of inducing or stimulating growth of tissue or bone.

In some cases, the photo-initiator is a ruthenium-based photo-initiator, optionally provided with an electron acceptor.

In some cases, the method comprising irradiating by a light having a wavelength in the visible range the aqueous formulation comprising (i) at least one native non-modified protein, being optionally gelatin, having an abundance of tyrosine amino acids, (ii) a water-soluble photo-initiator, (iii) at least one electron acceptor and (iv) living cells, causing the tyrosine amino acids to form di-tyrosine bonds transforming said formulation into said biological scaffold.

Further provided is a method for inducing or stimulating cell or tissue growth or generation in vivo or ex vivo, the method comprising instilling or positioning a biological 3D printed cell-free or cell-containing scaffold according to the invention in a tissue or an organ, wherein said scaffold optionally comprises cells capable of recruiting host cell infiltration, promotion of tissue growth, and/or tissue regeneration.

In some cases, a method concerning a cell-free scaffold may comprise a step of seeding or treating the scaffold with living cells.

In some cases, the scaffold further comprises active agents that improve the condition of the subject, stimulate healing of an injured tissue or organ, facilitate hastened healing, or reduce pain.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-B. Quantification of gelatin hydrogel degradation, G10_LNCO3 (low cross linking) and G10_LNCO8 (high cross-linking). Degradation behavior of G10_LNCO3 and G10_LNC08 in (A) single-implanted (n=6-7) and (B) double-implanted animals (n=10). Mean+s.d, * p<0.05, ** p<0.01.

FIG. 2. Degradation trend chart of collagen sponge and ACM in vivo.

FIGS. 3A-D. Macroscopic appearance and construct weight. The explanted specimen appeared translucent, and the loop was clearly visible in the middle. The construct after 2 weeks (A) and after 4 weeks (B) depicted no optical differences. The weight of the constructs showed no significant difference over time in vivo between the 2- and 4-week group (C) and the degradation in vitro in 1.75 μg ml⁻¹ collagenase and PBS as a control over 14 d (D). Scale bar=5 mm (A), (B).

FIG. 4. Absorbance of several formulations, different in photo-absorbers components. Including gelatin solution, gelatin with Ru/SPS, gelatin with Ru/SPS and Tartrazine (TRZ) as a dye, and gelatin with Ru/SPS and Phenol red (PR) as a dye (red). Irradiation intensity of DLP printer in the relevant wavelengths.

FIG. 5. Temperature history dictates different gelation points between the cooling and heating stages, emphasizing the use of the lower gelation point to achieve the ink viscosity requirement for DLP bioprinting. Temperature ramp test for gelatin type B at various wt % resulted in a hysteresis loop. The lower gelation point was measured for the cooling stage compared to the heating stage.

FIG. 6. Schematic presentation of the DLP printing process for non-modified protein-only ink. The procedure commences with the design of a CAD file, subsequently fed into slicing software that translates the 3D CAD design into a sequence of 2D patterns. These patterns are projected onto the photocurable ink, directing the initiation of a photo-chemical di-tyrosine reaction at specific locations as dictated by the pattern.

FIGS. 7A-D. Cure depth analysis enables a deeper understanding and better customization of the printing parameters. (A) Cure depth measurements for ink formulation without dye for two light intensities (3 reps). (B) Linear trends were observed for each formulation's cure depth, which correlates with the natural logarithm of the energy (N=3). (C) Calculated cure depth was determined for a range of light intensities as a function of exposure time. (D) XY-resolution test shown by area printed/theoretical (%) for each formulation at various exposure times were evaluated for each formulation (without dye, with Tartrazine, and with Phenol red). 100% resemblance is shown as a black line. All error bars represent confidence intervals (95%).

FIG. 8. Detection of di-tyrosine bond formation via fluorescence spectroscopy. Emission spectra (excitation at 285 nm) of a thin film of the ink formulation before and after irradiation. An increase in emission intensity around 400-410 nm post-irradiation indicates di-tyrosine bond formation. The broad peak observed is attributed to the complex molecular structure of gelatin, with its diverse amino acid composition and the heterogeneous environment of tyrosine residues within the protein matrix.

FIGS. 9A-G. The suggested formulation utilizes only unmodified gelatin, allowing for relatively high protein concentration DLP printing of objects with different complexities. (A) Y-shaped pipes with two different colorant solutions, (B) stretchable woodpile, (C) Anatomical heart model, (D) Phenol red has the unique property of pH-dependent color change, (E) stretchable network-shaped printed hydrogel, (F) different geometrical objects, and (G) Published works about DLP printing of proteins (full references table in the Supplementary material).

FIGS. 10A-B. Mechanical properties examination. (A) stress-strain curve for printed discs (8 reps); (B) oscillatory frequency sweep (OFS) results for printed discs (5 reps). Both storage and loss modulus remained constant for frequencies between 0.1-2 Hz. Error bars represent confidence intervals (95%).

FIG. 11. Oscillation Amplitude Sweep (OAS) results for printed discs (N=5). Based on these results, the LVER was identified, and a shear stress of 1 Pa was selected for OFS tests. Error bars represent confidence intervals (95%).

FIG. 12. Tailoring mechanical properties of the printed object by design. A series of woodpile designs were tested to evaluate the effect on compressive modulus. Smaller gaps between printed bars caused increase of the compressive moduli.

FIGS. 13A-D. Chondrocyte viability and growth within 3D printed cell-seeding constructs (Ru/SPS-0.8/8 mM). Chondrocytes were seeded onto the 3D printed scaffolds in a gelatin solution and grown in cell culture media for up to 7 days. (A) Bright-field microscopy images show cell attachment both on the surface (i, ii) and within the interior voids (iii, iv). (B) Cells within the constructs are shown by H&E histological staining. (C) Cell viability assay demonstrates cell survival and proliferation 5 and 7 days following chondrocytes seeding onto the printed scaffold, N=1, triplicate. Error bars represent standard deviation. (D) Overlayed microscopy image of Live (green)/Dead (red) dual staining performed with FDA/PI shows high cell viability rates.

FIGS. 14A-D. Chondrocyte viability and proliferation seeded in 3D printed scaffolds (Ru/SPS-0.8/8 mM). (A) Live cells evaluated by FDA fluorescence signal shows a significant increase in cell viability and proliferation at 7 and 14 days post-printing, N=1, triplicate. Error bars represent standard deviation. (B) Ki67 immunohistochemical staining of the seeded scaffolds, demonstrating cell proliferation on the printed construct. (C-D) Histological staining shows typical cartilage morphology, including lacunae and dense ECM deposition, following 8 weeks of in-vitro differentiation.

FIGS. 15A-F. Chondrocyte viability and proliferation in 3D printed constructs (Ru/SPS—0.8/8 mM) using cell-laden approach (Asiga printer-405 nm). (A, B) Bright-field microscopy images of chondrocytes embedded within woodpile-shaped constructs. (C, E) Cell viability assay results for chondrocytes subjected to 18-minute and 12-minute print times, respectively, assessed at 24, 48, 72, and 96 hours post-printing. (D, F) Trypan blue cell counting data confirmed chondrocyte viability and proliferation after printing for 18-minute and 12-minute durations, respectively. Chondrocytes were suspended in the bioink formulation, 3D printed, and cultured in cell culture media. All experiments N=1, triplicate. Error bars represent standard deviation.

FIGS. 16A-D. Chondrocyte viability and growth within 3D printed cell-laden constructs (Ru/SPS—0.8/8 mM). Chondrocytes were suspended within the bioink formulation and 3D printed. The woodpile-shaped printed constructs were grown in cell culture media for up to 7 days. (A, B) Bright-field microscopy images show embedded chondrocytes within the constructs presenting typical viable morphology. (C) MTS assay at day 7 post-printing demonstrates viability, N=1, triplicate. Error bars represent standard deviation. (D) Overlayed microscopy image of Live (green)/Dead (red) dual staining performed with FDA/PI shows high cell viability rates.

FIGS. 17A-B. Chondrocyte viability and proliferation in 3D printed constructs (Ru/SPS—0.8/8 mM) using cell-laden approach (Mono printer-460 nm). (A) Chondrocyte viability and proliferation evaluated by FDA fluorescence staining at 1, 2, and 7 days post-printing for constructs with an 18.5-minute print time. (B) FDA fluorescence staining demonstrates chondrocyte viability and proliferation in 3D constructs at 7 and 14 days post-printing. All experiments were performed N=1, triplicate. Error bars represent standard deviation.

FIGS. 18A-E. Chondrocyte viability and proliferation in 3D printed constructs (Ru/SPS—1.5/15 mM) using cell-laden approach (Mono printer-460 nm). Growth in serum-containing medium and stained with Calcein Propidium Iodide for viability assessment after 3 (A), 9 (B), 15 (C), and 21 (D) days, also with serum-free medium (E).

FIGS. 19A-C. Chondrocyte viability and proliferation in 3D printed constructs (Ru/SPS—1.5/15 mM) using cell-laden approach (Mono printer-460 nm). Constructs were grown in serum-containing medium and stained with Calcein Propidium Iodide for viability assessment after 21 days in serum-containing medium (A), and in serum-free medium (B). Cell attachment and growth demonstrated by Phalloidin staining of Actin filaments after 10 days in serum-free medium (C).

### DETAILED DESCRIPTION OF EMBODIMENTS

### Results

### Ink Formulation and Optimization

Gelatin, the partial thermally and chemically degraded product of collagen is highly biocompatible, biodegradable, low immunogenic, and low-cost material. It is therefore highly attractive for use in clinical applications in the field of tissue regeneration. Gelatin preserves critical characteristics of collagen, the major extracellular matrix (ECM) component. The amino acid sequence similarity between collagen and gelatin defines its value for biological signaling, including for example integrin binding signaling and molecular attachments which are considered crucial for tissue regeneration. The polymer-type behavior of gelatin, either through spontaneous gelation in physiological environment or via chemical cross linking makes it good candidate in cases where 3D structures are required to promote specific biological characteristics.

As with any bio-degradable material intended for clinical application, understanding the mechanism of degradation in-vivo is an important factor in the design and evaluation of performance. Degradation paths of gelatin, in practice, can be related to the degradation of collagen.

Triple-helix collagen is quite resistant to proteolytic degradation. Therefore, degradation of collagen in vivo requires specific types of proteolytic enzymes related to the family of matrix metalloproteinases (MMPs) and Cathepsin. MMPs for example, locally unwind the triple-helical structure of collagen before hydrolyzing the peptide bonds. There are at least 25 members of the MMP family that are commonly classified based on substrate specificity and molecular structure into groups. These include the collagenases (MMP-1, MMP-8, MMP-13, and MMP-18) and gelatinases (MMP-2 and MMP-9). Collagenases are released by several cells in the body including macrophages, fibroblasts, neutrophils and tumor cells. In chronic pressure ulcers, an influx of neutrophils releases MMP-8, a potent collagenase. The cathepsins are a group of small proteinases that are most active at acidic pH and are usually characterized as lysosomal proteinases. The cathepsin family can cleave multiple components of the ECM. In vitro studies have shown that several members of the cathepsin family are able to cleave the fibrillar collagens. The cathepsin family consists of at least 16 members and can be subdivided into three distinct groups. Most of the cathepsins are lysosomal proteinases.

In the case of gelatin that is used as implantable component or treatment, the triple helix of the native collagen is already opened, therefore can expect that gelatin is more sensitive than intact collagen to proteolytic degradation by collagenases and by other proteolytic enzymes.

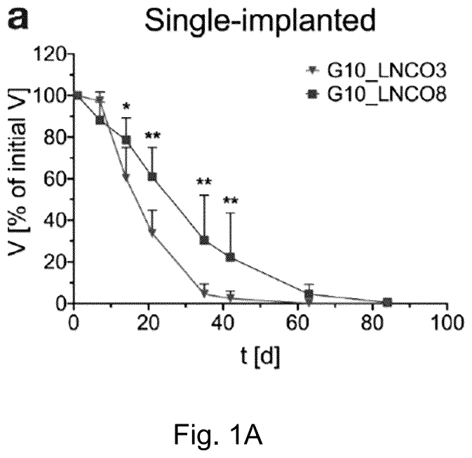

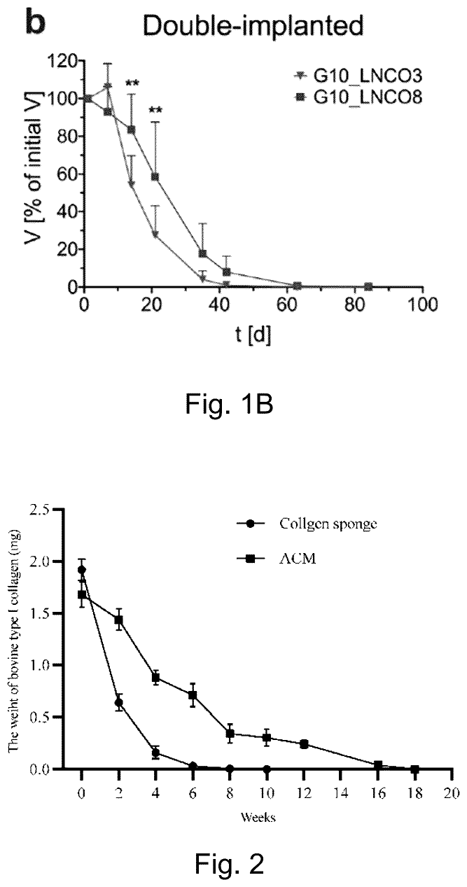

The expected degradation time of a specific gelatin-implant depends on number of variables, including the location of the implant and the level of covalent cross linking. In one study for example, the degradation rate of cross-linked gelatin implant was assessed in-vivo. Multimodal imaging revealed that the number of covalent net points correlates well with degradation time, extending the complete degradation by 50-100%, from about 40 days to 60-80 days (FIG. 1). The degradation was correlated with the activity of MMPs.

As the mechanism of gelatin degradation is related to the native collagen degradation, a reference to collagen degradation studies is also relevant. For example, the impact of level and type of cross linking was evaluated in vivo.

In this model, the degradation of cross-linked collagen sponge (Type I) and Acellular matrix (ACM) were compared in a rabbit model (FIG. 2). Clearly the type and the structure of the implant had a significant effect on the rate of degradation, extending the complete degradation of the highly-cross-linked ACM to 16-18 months compared with 6 months of the collagen sponge.

The observed degradation rates of commercial collagen-based products extend from a couple of weeks to over two years (Table 1). The reason for this huge difference stems from the different sources, production process, cross linking and specific model for testing.

TABLE 1
Degradation in-vivo of commercial products based on
different tissue sources and differently crosslinked

| | | | | Degradation |
|---|---|---|---|---|
| | | | | in vivo |
| | Supplier | Crosslinking | Species | (weeks) |
| BioGide | Geistlich Biomaterials | | Porcine skin | 2-4 |
| BioMend | Sulzer Medica; now Zimmer | Glutaraldehyde | Bovine tendon | 4-8 |
| | Biomet | | | |
| BioMendExtend | Sulzer Medica; now Zimmer | Glutaraldehyde | Bovine tendon | 18 |
| | Biomet | | | |
| TutoDent | Tutogen | — | Bovine pericard | 8-16 |
| Ossix | Colbar R&D ltd.; now | Enzymatic/ | Bovine tendon | >24 |
| | datumdental | carbohydrate | | |

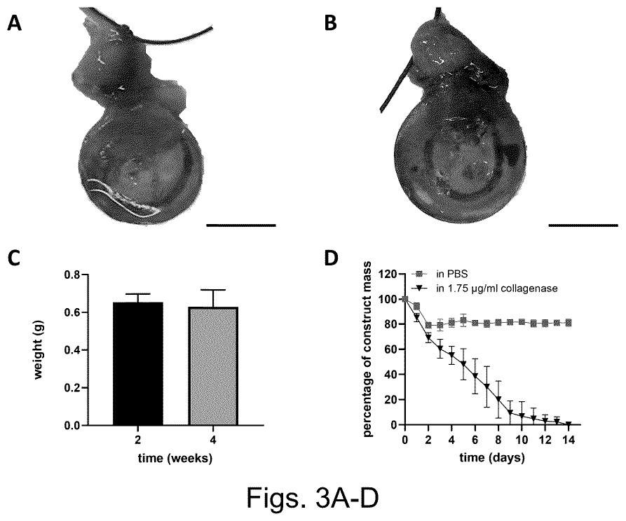

An important observation in this context is the difference between degradation in-vitro and in-vivo. In a study to test the degradation of scaffolds fabricated of gelatin-methacrylate (Gel-Ma), The authors evaluated the degradation of Gel-Ma scaffolds in-vivo, after implantation as compared to degradation in-vitro in the presence of collagenase (FIG. 3).

The degradation in-vitro was significantly faster than in-vivo, with complete digestion after 2 weeks while the scaffold implanted in the animals was completely stable for 4 weeks.

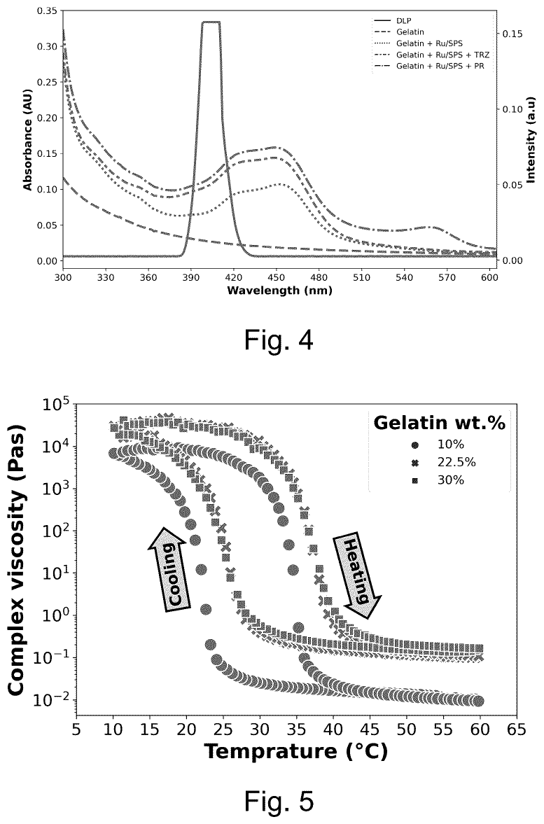

Gelatin is considered a thermo-responsive material, as it undergoes a reversible sol-gel transition between solution (known as “sol” phase) and amorphous solid (known as “gel” phase). This process results in significant changes in the viscoelastic properties of the material, making it an important factor in view of printability and workability since the mobility of the reacting molecules is an essential factor for the polymerization process. A series of gelatin printing compositions was prepared to test the suitability for the printing process by irradiating samples in a mold at 405 nm. It should be noted that although the peak of the ruthenium complex is at 452 nm, it has sufficient absorption at 405 nm, which is common for commercial printers (FIG. 4). Upon irradiation, it was found that the polymerized gelatin, at concentrations below 10 wt. % gelatin, did not maintain its original shape after polymerization. The structural integrity depends on the crosslinking density, which can be evaluated by determining the cross-over point. It is the intersection between the storage and loss moduli, as measured by Oscillation Amplitude Sweep (OAS) tests. The results showed a significant increase in the crossover point from 300 Pa for 10 wt. % gelatin to 2800 Pa for 22.5 wt. % gelatin, indicating a general trend of enhanced crosslinking density and mechanical stability at higher concentrations.

Conversely, formulations with gelatin concentrations exceeding 15 wt. % required high temperatures to be printed due to the high viscosity at room temperature. At a gelatin concentration of 30 wt. %, a printing temperature of around 45° C. was found to be optimal.

The elevated temperature during printing (45° C.) causes the denaturation of the gelatin, resulting in a random coil tertiary structure. As gelatin is a linear protein, its tertiary structure is inherently simple. Importantly, the primary structure of gelatin remains stable up to 70° C., ensuring its integrity throughout the entire printing process.

Notably, the ink formulation also included SPS, which undergoes thermal activation above 70° C. for an extended duration, and therefore, a practical upper limit of 30 wt. % gelatin was selected for the subsequent printing experiments. Preliminary experiments were conducted to optimize the Ru/SPS concentrations, as the ratio between the two components was maintained at 1:10, as described in several publications.

The rheological properties of three gelatin solutions are presented in FIG. 5.

As expected, higher concentration led to higher viscosity. Interestingly, a hysteresis loop was observed during the measurements, which involved two measurement stages: heating from 10° C. to 60° C., followed by a cooling stage back to 10° C. Because of this hysteresis, the gelatin solutions were prepared at a high temperature, 65° C., while the printing experiments were performed at 50° C.

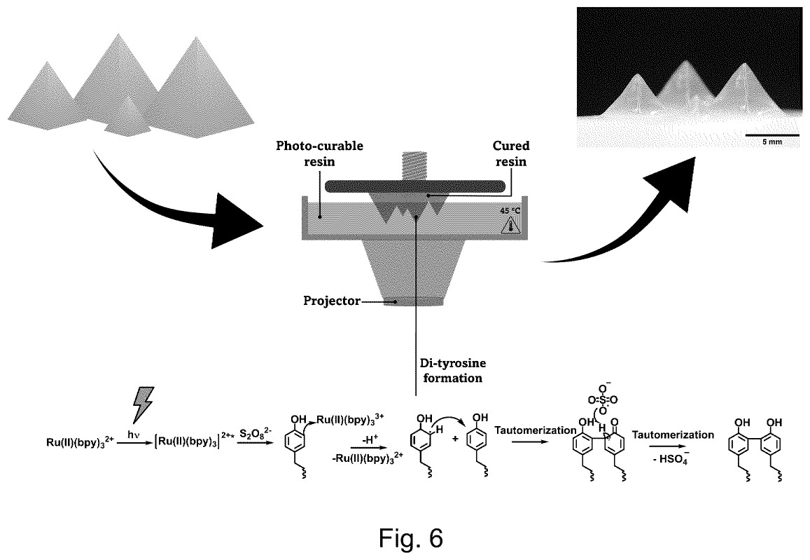

The printing experiments were performed according to the scheme presented in FIG. 6. In essence, the protein solution contained the Ru/SPS initiator, placed in the heated DLP vat, and irradiated at 460 nm via a layer-by-layer process, according to the sliced CAD file. The resulting object is a crosslinked hydrogel rinsed off with water (80° C.) to remove all unpolymerized material.

### Optimization of Printing Parameters

When fabricating each layer by the DLP process, the depth to which the light beam penetrates the resin critically governs each voxel dimension. As established by Jacobs, the cure depth can be expressed by the relationship of Eq. 1:

Cd=Dₚ·ln(E/E_{c}), Eq. 1:

wherein Cd—cure depth, Dp—penetration depth, E—energy dosage, Ec—critical energy. It is important to note that the energy dosage is the product of light intensity, given in units of mW cm⁻², and the exposure time in seconds.

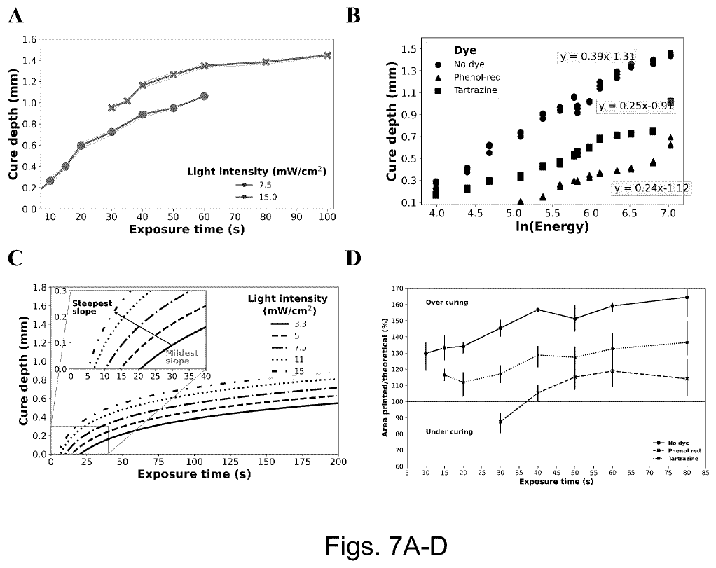

The cure depth of various formulations was evaluated by irradiation within the DLP vat at given energy doses and by measuring the thickness of the cured layer with an optical microscope. FIG. 7A shows the cure depth as a function of exposure time for two light intensities. As seen, increasing the exposure time led to increased thickness for both light intensities, leveling off at the higher intensity and long irradiation duration. As shown in FIG. 7B, the cure depth of formulations without a dye and with Phenol red and Tartrazine were linearly dependent on the natural logarithm of the energy. According to Jacob's working curve, the curve's slope represents the formulation's penetration depth, while the y-intercept corresponds to the critical energy required for polymerization. Interestingly, the critical energy for the composition without a dye was 28.8 mJ cm⁻², compared to 38.1 mJ cm⁻² and 154.4 mJ cm⁻² for compositions containing Tartrazine and Phenol red, respectively.

The curing depth was determined for several theoretical light intensities, and the results were plotted as a function of exposure time (FIG. 7C). Utilizing the linear equation derived from the working curve, these trends were calculated, with a fixed light intensity chosen for each trend. This analysis enabled the identification of a specific light intensity that would allow easy tuning of the cure depth while maintaining short printing times. As seen in FIG. 2C (inset), at high light intensities, the slopes are steep, thus predicting significant changes in cure depth upon minor changes in exposure time. Conversely, lower intensity levels require printing times that are too long. Therefore, we selected irradiation of 7.5 mW cm⁻² for which the cure depth is not very sensitive to irradiation time, and yet it enables printing in short durations, ˜40 seconds. Confining the polymerization area to small spots is crucial to achieve a high dimensions similarity between the CAD file and the printed object. This can be accomplished by employing photo absorbers (dyes) that can absorb a certain amount of light without causing polymerization. For bioprinting applications, selecting appropriate dyes becomes crucial, as they should absorb the specific wavelength and yet be non-harmful to living systems. Phenol red emerges as a promising candidate for the intended application due to its absorption characteristics around 400 nm and its frequent use in cell culture media. It has a very low toxicity, excellent stability within living systems, and minimal interference with biological processes. Tartrazine, another pertinent dye, has been employed in diverse cell-related systems, serving as a coloring agent in food and cosmetic products. The results shows that formulations containing these dyes exhibit stronger absorbance at the relevant wavelength of the printer (405 nm), making them suitable choices for investigation as potential resolution enhancers in the ink formulation. The UV-Vis spectra of the various solutions are presented herein, along with the printer's irradiation peak and intensity. As described above, the critical energy of polymerization was the highest for Phenol red, 154.4 J cm⁻². Thus, it should be very effective in controlling resolution, and therefore, the subsequent experiments were performed with this dye.

Different dyes affect the resin's curing depth in various ways due to their distinct absorption characteristics at the DLP printer's operating wavelength. As shown, phenol red displays higher absorption in the relevant wavelength range, which helps to confine the polymerization reaction to the selected pixel and reduce light scattering effects. Additionally, energy transfer can occur within the system, primarily from the Ruthenium complex to phenol red. This further contributes to a more localized reaction, preventing over-curing and resulting in a higher resolution. In contrast, the Tartrazine formulation facilitates energy transfer in the opposite direction, from the dye to the Ruthenium complex. While this enhances the reaction, it may also lead to the over-curing of the fabricated object. Given that our main goal in adding a dye is to achieve higher resolution, phenol red was determined to be more suitable for our purposes. This preference is reflected in the critical energy value, representing the energy threshold required to initiate the reaction. Furthermore, such formulations are susceptible to extensive polymerization along the z-axis, resulting in over-curing in this dimension. The relationship between print speed and resolution presents a significant parameter in optimizing the printing process. Formulations with higher critical energy, such as the phenol red-based mixture used in this study, require longer exposure times. While beneficial for achieving higher resolution, this increased exposure time impacts the overall print speed.

The XY resolution was evaluated by printing rectangles and comparing their dimensions to the corresponding CAD file. This assessment involved measuring the area of each printed rectangle using ImageJ software. FIG. 7D shows the % area printed/theoretical area for each formulation at various exposure times, while 100% is defined as the highest resolution. Above 100% is termed overcuring, and below 100% is termed under-curing. In general, as expected, the higher the exposure time, the larger the printed area compared to the designed area. The formulations without dye and with Tartrazine were always overcured (and at lower exposure time, did not cure at all).

In comparison, the formulation containing Phenol red was either over-cured or under-cured, while at 40 seconds of exposure, the ratio was 100%, meaning excellent resolution. Therefore, the subsequent printing experiments were performed at the same irradiation energy (300 mJ cm⁻²). These experiments were under irradiation intensity, meaning that in view of energy, the irradiation time can be shortened by increasing the intensity.

### 3D Printed Models

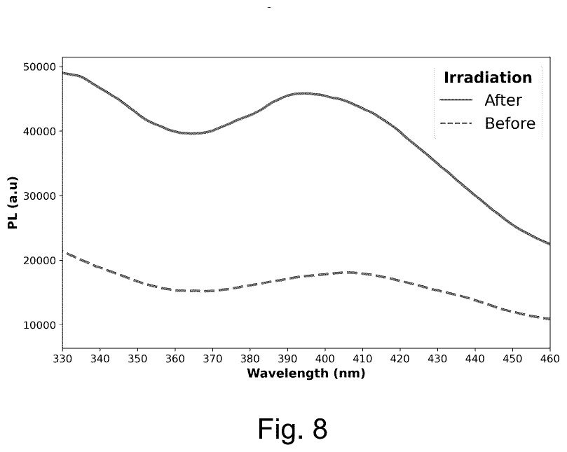

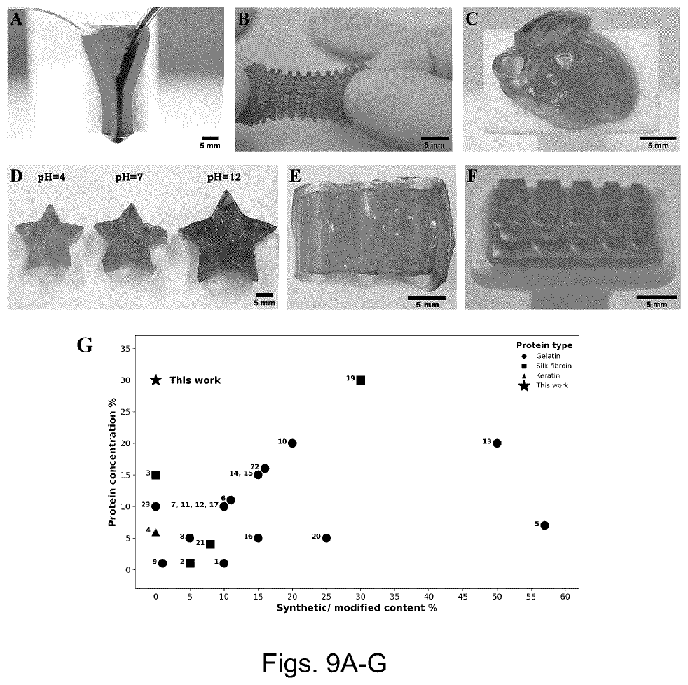

By using a gelatin concentration of 30 wt. %, simple shapes could be 3D printed, e.g., pyramids shown in FIG. 6. We conducted fluorescence spectroscopy analysis to further validate the formation of di-tyrosine bonds and distinguish between physical gelation and photo-crosslinking. The resulting spectra (FIG. 8) show an increase in emission intensity around 400-410 nm after irradiation, consistent with di-tyrosine bond formation. However, it is important to note that due to the complex nature of gelatin, which contains tyrosine residues within a large molecule with many chemical entities, the fluorescence spectrum presents a broad peak rather than a sharp, easily quantifiable signal known for small molecules. This complexity limits the ability to perform precise quantitative analysis of the crosslinking reaction based solely on fluorescence intensity differences. These results, combined with the thorough washing protocol at 85° C., which dissolves any uncrosslinked or physically gelled gelatin, provide strong evidence for the successful photo-crosslinking of the gelatin-based ink. However, for more complex structures, it was found that it was necessary to decrease the gelatin concentration. After evaluating various concentrations, a 22.5 wt. % was selected, enabling printing structures as shown in FIG. 9. Interestingly, the printed hydrogel structures were stretchable, as seen in FIG. 9B. Another noteworthy feature of the printed gelatin structures is their response to pH change, in view of swelling and color, which makes the object classified as 4D printed. The printed structures were immersed in aqueous solutions having various pH values. As seen in FIG. 9D, the objects swell with the increase in pH, with higher pH values leading to more substantial swelling. The swelling increases by ˜140% while immersed at pH 12 compared to pH 4. This phenomenon results from the deprotonation of the gelatin (type B, isoelectric point of 4.5) as the gelatin molecules are crosslinked.

The use of Phenol red as a dye also enables color and response while the pH is the stimuli, as shown for other 4D printed objects. At pH 4, the hydrogel containing Phenol red displayed a yellow color, and as the pH increased, the hydrogel transitioned to a solid red appearance at pH 12. This pH-dependent color shift further highlights the potential use of colorants, including PR, in bioprinting applications at which pH monitoring or visual detection is essential, such as wound healing applications.

Once the optimal composition and printing parameters are defined, printing hydrogel structures with high complexity is realized. As presented in FIG. 9, these printing compositions enabled the fabrication of overhanging structures and objects with embedded, open tubings, which are essential for bioprinting implants with blood vessels.

The addition of photo-absorbers plays a crucial role, particularly in fabricating tubular structures. These additives absorb some of the irradiated light, effectively confining the crosslinking reaction to smaller, more precisely defined areas. This confinement is especially important along the z-axis when creating open tubes. In the z-axis, the photo-absorbers enable the polymerization to occur up to a specific depth while leaving subsequent layers unpolymerized. This controlled cure depth is essential for forming the open part of the tube. Without photo-absorbers, the light could penetrate too deeply, resulting in solid structures rather than hollow tubes. The use of photo-absorbers must be combined with careful optimization of light irradiation energy. This optimization ensures that the resin absorbs enough energy to initiate and sustain the crosslinking reaction while preventing overcuring, particularly along the z-axis. Achieving the right balance between photo-absorber concentration and light energy is crucial. If there is too little energy, the polymerization process may be incomplete, resulting in mechanically weak structures. Conversely, excessive energy can cause overcuring and compromise the structural integrity, particularly when creating hollow features. This delicate balance between photo-absorbers and light energy allows for precise control over the geometry and the mechanical properties of the printed tubular structures.

It's noteworthy that the majority of existing research in DLP printed hydrogels commonly uses proteins with covalently bonded polymerizable groups or compositions, including synthetic monomers. Conversely, prior published works employing protein-only formulations typically encompass much lower protein concentrations than our current invention, a feature that can significantly impact biocompatibility aspects. FIG. 9G presents the published work with DLP printing of proteins, showing that there are only a few non-modified ones, while we use the highest reported concentration of non-modified protein.

### Mechanical Properties of Printed Structures

To fabricate the 3D cartilage scaffold, it is essential to tailor the material's mechanical properties for soft tissue to facilitate the growth and proliferation of cells, which later degrade as the cells differentiate and form grown cartilage. The mechanical characterization of the printed structures followed two main paths. Initially, the focus was on how the hydrogel behaves under unconfined compression.

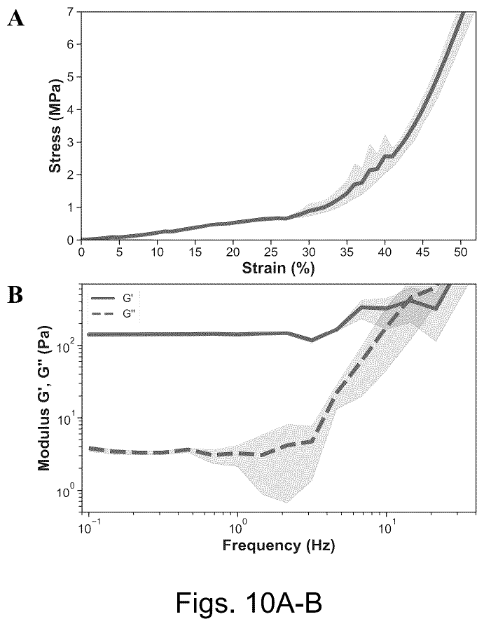

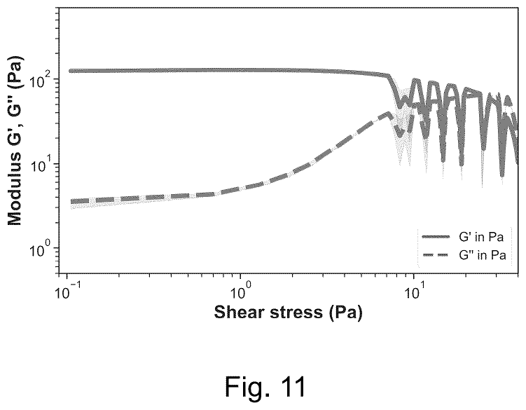

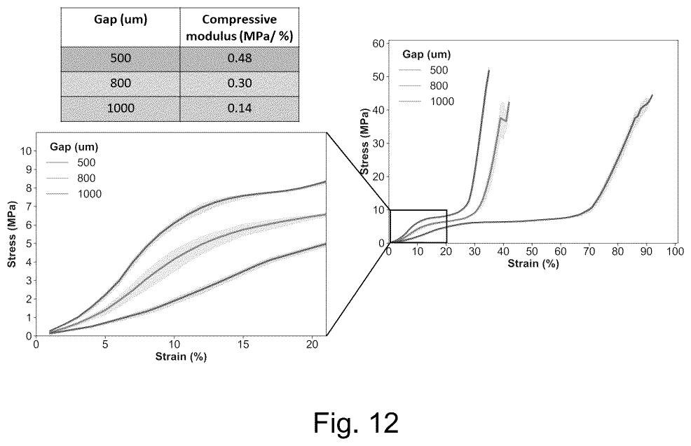

Compression tests were chosen for mechanical characterization as they are generally considered more accurate for describing hydrogel mechanical properties. Eight printed discs underwent a compressibility test, revealing a compression modulus of 31 kPa (FIG. 9A). The printed objects have a modulus similar to soft tissues and within the range of printed GelMA (chemically modified gelatin, with methacrylate groups). As a result, it is a suitable candidate to serve as a spatial guiding scaffold for tissue growth. The second approach involved oscillatory measurements, including amplitude and frequency sweeps. The storage and loss moduli obtained from the measurements at the linear viscoelastic region are shown in FIG. 10B and FIG. 11. The storage modulus was 132.97±1.23 Pa, while the loss modulus was 99.02±1.25 Pa. These values are consistent with the trends observed in the earlier compression tests discussed in this section, providing a coherent characterization of the material's mechanical properties.

### Biological Studies

Cell adhesion and growth onto and within the gelatin-based 3D constructs were evaluated by examining two cell fabrication techniques: (1) Cell-seeding onto the woodpile-printed gelatin scaffold and (2) printing compositions that contain living cells during the printing process (cell-laden). In the first approach, the cells are not exposed to light irradiation, and the cell growth occurs upon attachment on the woodpile bars of the scaffold and continues to the voids. In the second approach, the cells are exposed to light irradiation and embedded within the gelatin matrix as the printing proceeds. Therefore, the cell growth, in this case, occurs within the polymeric network and continues to the voids between the woodpile bars. Cell survival may be impacted by both deficient adhesion conditions and irradiation. However, we use light within the visible spectrum, which should not be harmful to the cells.

### Cell-Seeding Approach

Using this approach, it was found that chondrocytes seeded onto the 3D printed gelatin scaffolds showed adhesion, survival, and proliferation over 7 days of immersion in culture media.

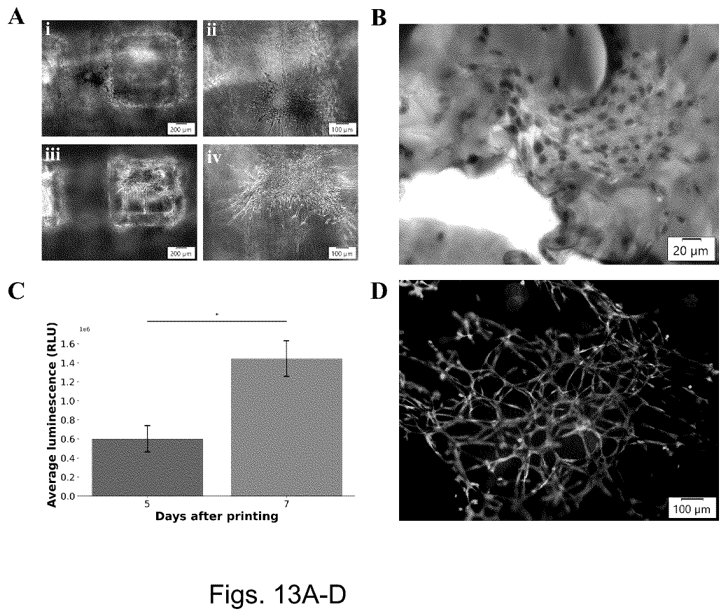

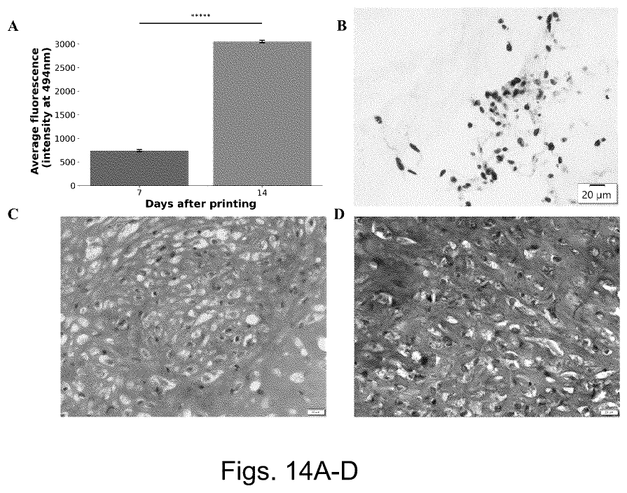

Cultured human nasal chondrocytes were suspended in 8% gelatin to enhance initial adhesion and then applied onto the printed scaffolds, followed by incubation in growth media. Bright-field imaging and H&E histological analysis showed that chondrocytes adhered to the surface of the scaffold, as shown in FIG. 13A and FIG. 13B. A cell viability assay indicated that viable cells were present at days 5 and 7 post-seeding, as shown in FIG. 13C. The viability of the cells was also confirmed by Live/Dead staining, depicted in FIG. 13D and FIG. 14. Additionally, Ki67 immunostaining confirmed the presence of proliferating chondrocytes distributed throughout the scaffold after 7 days, as shown in FIG. 13. Moreover, printed constructs were cultured in-vitro under chondrogenic conditions for 8 weeks. Alcian blue and Safranin O staining demonstrated cartilage tissue formation, indicating ECM deposition as show in FIG. 14C-D. These results demonstrate that the 3D printed gelatin scaffolds effectively support chondrocyte attachment, growth differentiation and maturation.

### Cell-Laden Approach

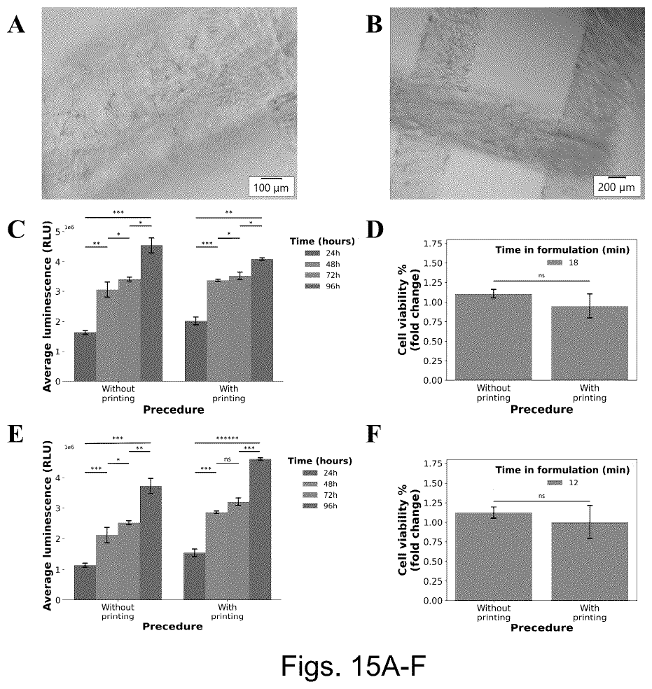

In this part of the research, the cells were dispersed within the printing formulation. This was followed by a 3D printing process in which the cells were exposed to light irradiation and the mechanical forces occurring during printing. In general, it was found that chondrocytes remained viable, and the cell growth continued within the printing composition and after the printing process. Microscopy imaging showed the presence of embedded chondrocytes within the cell-laden constructs 4 days after printing (FIG. 15A and FIG. 15B). To evaluate the effect of the duration of the printing process on cell survival and proliferation, the printing was performed for 12 or 18 minutes, and the cells were subsequently cultured for viability analysis. The cell viability assay was performed at 24, 48, 72, and 96 hours, and no significant difference in viability was observed between the two printing durations, indicating similar proliferation capacity of the two groups (FIG. 15C and FIG. 15E). Trypan blue staining at day 7 further confirmed that cell viability and proliferation were maintained after the printing process, demonstrated by the fold change from the time zero (original cell culture) (FIG. 15D and FIG. 15F). It is worth noting that the fold change of all tested groups is close to 1 or higher, providing further confirmation that the cells were able to continue proliferation after the suspension in the ink formulation and the printing procedures.

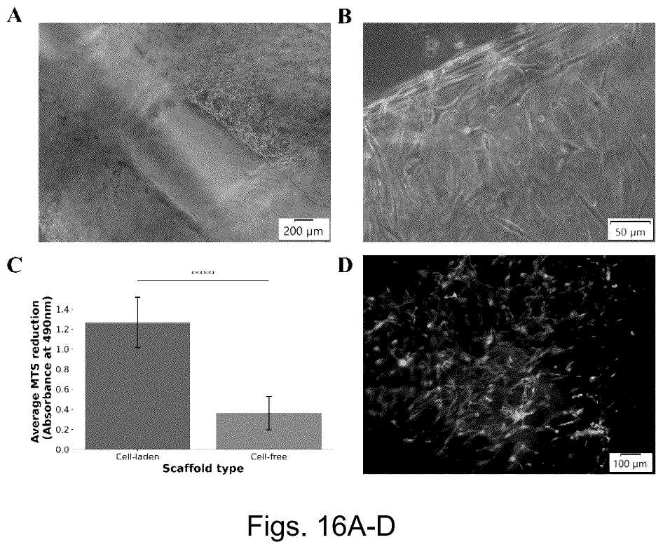

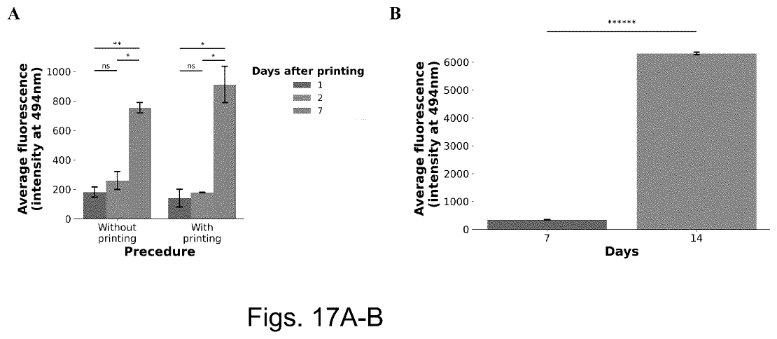

To ensure high cell survival rates, minimizing phototoxicity and maintaining a consistent temperature of 37° C., particularly during extended printing sessions, is crucial. To overcome these challenges, we used the ‘MONO printer,’ which operates at a wavelength of 460 nm for cell-laden bioprinting experiments. Woodpile-shaped cell-scaffold structures were printed in cycles lasting 18 to 20 minutes (FIG. 16). As we demonstrated using the “ASIGA printer” (FIG. 14C-D), cells suspended in the ink before and after the printing process, were collected and cultured for 7 days (FIG. 17A), display similar viability and proliferation rates (FIG. 15C and FIG. 15E). After printing, the constructs were incubated to promote cell growth for up to 14 days. Microscopy imaging revealed that chondrocytes were embedded within the scaffold two days after printing, as shown in FIG. 16B. An MTS metabolic assay demonstrated cell viability within the scaffold at day 7, as illustrated in FIG. 16C. Additionally, cell viability was confirmed by Live/Dead staining at both 7 and 14 days post-printing (FIG. 16D and FIG. 17B).

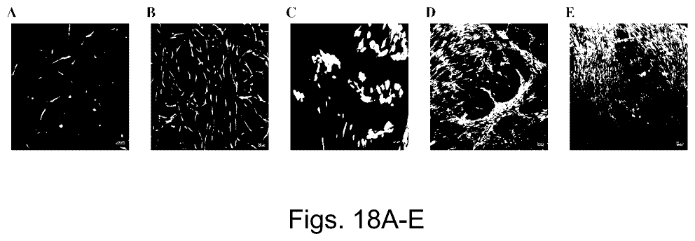

While FIGS. 14-17 demonstrate cell growth and proliferation within 3D-printed cell-laden constructs using the Ru/SPS 0.8/8 mM formulation, FIGS. 17-18 demonstrate cell viability, growth and proliferation within constructs formulated with a higher concentration bioink (Ru/SPS 1.5/15 mM).

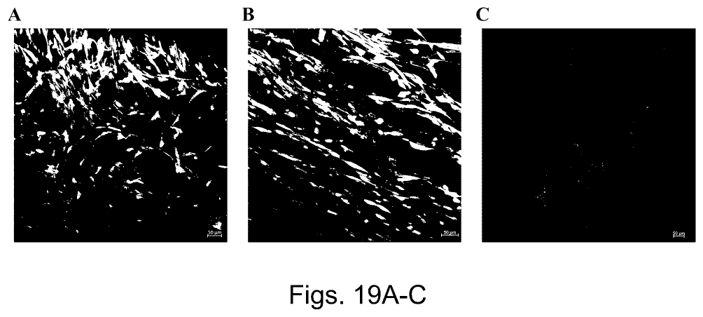

Chondrocyte viability, proliferation, and growth were observed under incubation in serum-containing medium (FIGS. 18A-D, FIG. 19A) as well as in serum-free medium (FIG. 18D, FIGS. 19B-C).

### CONCLUSION

In this work, a pristine gelatin solution was photopolymerized by Ru/SPS to fabricate 3D objects by DLP printing. Structures with different architectural complexities were successfully printed, including overhangs, open tubes, and detailed features, including hollow tubes. Ru/SPS transforms the dissolved gelatin into a crosslinked network by generating di-tyrosine bonds between the residues of two tyrosine amino acids. Therefore, the bioink is low-cost, as no chemical modification of the gelatin is required. This also decreases the risk of antigenicity responses for future biological applications. Additionally, we harness a new functionality of the dye Phenol red, which is used in cell culture media as a visual pH indicator; here, it functions as a photo-absorber pH visual indicator.

We demonstrated that complex 3D objects could be achieved using a typical DLP printer, reaching a high protein content of 30 wt. % gelatin.

The printed object showed a compressive modulus of 31 kPa, which is in the region of soft tissues, relevant for the potential implantation use of the gelatin-based printed objects.

The biological results shown in the current study demonstrate the feasibility of using visible light-based 3D printing approaches to fabricate gelatin constructs for tissue engineering applications, specifically for engineering cartilage tissue. The results indicate that the 3D printed gelatin scaffolds support chondrocyte adhesion, survival, and proliferation, as evidenced by the cell-seeding experiments. Furthermore, the cell-laden printing approach, in which chondrocytes were incorporated directly within the gelatin formulation prior to printing, also enables maintaining cell viability and proliferative capacity for the tested durations.

The degradation profile of our crosslinked gelatin scaffold is critical to its potential biological applications. Based on previous studies with similar compositions, we estimate the degradation time of our crosslinked gelatin to be 3-4 weeks. This estimation is derived from the known two-week degradation time for a similar but lower concentration gelatin ink. The higher gelatin concentration is expected to extend this degradation period. It is important to note that the degradation of native gelatin in physiological environments can vary. Significantly, our crosslinked gelatin's estimated 3-4-week degradation time aligns well with the time scale for mature cartilage growth. This matching allows for the potential tailoring of growing tissue's 3D structure with a gelatin-based scaffold, opening up possibilities for tissue engineering applications. The rationale of this study is to fabricate a scaffold with dual characteristics. On one hand, it has sufficient shape fidelity to fabricate complex 3D structures. On the other hand, its degradation profile is relatively short-term, enabling replacement by the cellular components (cells and ECM). The mature scaffold within the body is expected to gain mechanical stability from the biologically engineered tissue.

Overall, the formulation and printing process presented herein have the potential to revolutionize the use of non-modified protein-only bioinks for the fabrication of intricate 3D objects. This development is anticipated to open up new research avenues in biomedical engineering, making it possible to create previously unattainable structures with high fidelity and precision while using pristine proteins.

### Experimental Section

### Materials

Gelatin from porcine skin—type B, [Ru(bpy)₃]⁺², Tartrazine, and sodium phosphate (dibasic and monobasic) were purchased from Sigma Aldrich. Sodium persulfate (SPS) was purchased from Holland Moran, Israel. Phenol red was purchased from Acros Organics.

### Bioink Preparation

Phosphate buffer saline (PBS) was prepared according to standard protocol. Gelatin (30 or 22.5 wt. %) was dissolved in PBS and heated to 50° C. while stirring. Concentrated solutions of 40 mM [Ru(bpy)₃]⁺² and 400 mM SPS were diluted to a final concentration. Solutions of 0.1 wt. % were made for each photo-absorber and were diluted to 0.01 wt. % in the final ink composition (0.28 mM of Phenol-red and 0.19 mM of Tartrazine).

Fabrication of 3D structures in Mold

UV-curable solution was poured into a mold and irradiated for 60 sec with a 405 nm UV torch (32 DC V, 8 A).

### DLP Printing

A predesigned CAD printed model was generated using a DLP printer (Asiga Max X35, Australia) equipped with a 405 nm LED. The printer's XY-axis resolution is 35 μm, while the maximum resolution on the Z-axis is down to 1 μm. The printing composition was poured into the designated heating bath heated to 50° C. Each printed layer was 100 μm thick and was cured for 35 sec with a light intensity of 7.5 mW cm⁻². The resulting printing rate is 1 cm/50 min (height). All printing experiments (excluding cell-laden ones) include a thorough washing stage. The vat was heated to 45° C. for printing without cells, followed by a post-printing washing stage until complete dissolution of the unpolymerized portion (water at 85° C. for 5-10 minutes). For the cell-laden printing experiments, the vat was maintained at 37° C. during printing, followed by a post-printing rinsing stage, until the full dissolution of the unpolymerized portion (PBS×1 with 1% Penicillin-Streptomycin and 0.1% Amphotericin B, at 37° C., 3-5 times, each for 5 minutes). Both scaffold types were then sequentially washed six times for 5 minutes each, in PBS×1 with 1% Penicillin-Streptomycin and 0.1% amphotericin B solution on a shaker at 37° C. The 3D objects STL files were obtained from Thingiverse, Anatomical heart by airforce (https://www.thingiverse.com/thing: 942464, license: CC-BY-SA), 3D Benchy by Creative-Tools (https://www.thingiverse.com/thing: 5293974, license: CC-BY-ND). All other STL files were designed with Autodesk Inventor software.

### Electron Microscopy

Scanning Electron Microscope (XHR-SEM) images were obtained using an extra-high-resolution scanning electron microscope (XHR Magellan 400 L).

### UV-Vis Spectrophotometry

UV-Vis spectrophotometry was used to quantify the ink components' absorption. Spectrophotometric measurements were recorded using a UV-spectrophotometer (UV-1800; Shimadzu, Japan). Absorption spectra of ink components (gelatin, gelatin with PI, and gelatin with PI and various dyes) were recorded in the spectral range of 300 to 800 nm with a resolution of 0.5 nm.

### Fluorescence Spectroscopy

Steady-state fluorescence measurements were performed on a HORIBA JOBIN YVON Fluoromax-4 spectrofluorometer with the excitation/emission geometry at right angles. A thin film of the ink formulation was applied to a quartz slide, and fluorescence measurements were taken before and after irradiation (excitation at 285 nm, emission spectrum recorded from 300 to 500 nm).

### Compression Test

For the compression test, discs (10 mm in diameter and 5 mm thick) were printed using bioinks and washed with water at 85° C. for 5 min. Each printed model's diameter and thickness were measured at three different areas, and the reported values are the averages.

Compression mechanical tests of the obtained discs were performed on the fully cured samples using an Instron universal testing machine (Model 3345, Instron Corp., Norwood, MA) equipped with a 500 N load cell. The compression test was conducted at 25° C. using 10 mm min-1 crosshead speed.

### Tensile Test

For tensile test, dogbone-shaped samples (15×5×3 mm) were printed using bioinks and washed with water at 85° C. for 5 min. Each printed model's length, width, and thickness were measured at three different areas, and the reported values are the averages. Tensile mechanical testing was conducted using an Instron Universal Testing machine (Model 3345, Instron Corp., Norwood, MA) equipped with a 500N load cell, at a rate of 10 mm min-1. The reported value is the average of the five printed models. Young's modulus was calculated using the slope of the linear portion of the curve, corresponding to the material's elastic region.

### Dynamic Mechanical Analysis

Discs with a nominal sample size of 10×5 mm (diameter×height) were printed to evaluate DMA properties. Rheometer (RheoStress HAAKE 6000) was used with a parallel plate geometry (P35 Ti L-60 mm) at 25° C. Oscillation Amplitude Sweep (OAS) was carried at a frequency of 1 Hz, and a range of shear stresses of 0.1-1,000 Pa was tested. The gap between the plates was set to fit 1.5 N.

After identifying the Linear Viscoelastic Region (LVER) from the OAS tests, the desired shear stress (Tau) for later assays was chosen. Oscillation Frequency Sweep (OFS) was carried at shear stress of 1 Pa for frequencies of 0.1-100 Hz. The gap between the plates was set to fit 1.5 N.

### Cell Isolation and Expansion

Tissue sample collection was approved by the Sheba Medical Center Ethics Committee (4745-17-SMC). All patients signed consent forms. Chondrocytes were isolated from nasal cartilage as previously described by the inventors. In brief, nasal cartilage was cut into 1-3 mm pieces, incubated with collagenase II for 12-14 hours, and washed with growth medium (DMEM/F12 with 10% fetal bovine serum and 1% pen-strep) following a 100 μm strainer filtration. After being centrifuged at 600×g for 8 minutes, the cells were cultured with growth medium replacement every 2-3 days until reaching a confluence of 80%.

Chondrocyte Seeding onto Printed Gelatin Scaffolds

Chondrocytes in passage two were trypsinized (0.25% trypsin-biowest) and washed with a growth medium. Following cell count, the supernatant was gently aspirated, and the pellet was suspended (30 μL of 8% gelatin at 37° C.) and seeded drop-wise onto the gelatin scaffolds at a cell concentration of 15-30×104/μL. The seeded scaffolds were incubated at 37° C. in a humidified atmosphere of 5% CO2 in air in a growth medium and replaced every 2-3 days.

Viability and Proliferation Analysis within Constructs:

RealTime-Glo™ MT Cell Viability Assay (Promega) was performed according to the manufacturer's instructions. The assay uses a cell-permeant luciferase substrate and a cell-impermeant oxidoreductase enzyme. This enzyme is only active in metabolically active cells. When the enzyme reduces the substrate, it produces a luminescent signal that is proportional to the number of viable, metabolically active cells. Luminescence was measured using a GloMax plate reader (Promega).

CellTiter 96® Non-Radioactive Cell Proliferation Assay (MTS) (Promega) was performed according to the manufacturer's instructions. The assay uses a tetrazolium compound called MTS (3-(4,5-dimethylthiazol-2-yl)-5-(3-is carboxymethoxyphenyl)-2-(4-sulfophenyl)-2H-tetrazolium). This compound bioreduced by cells into a colored formazan product that is soluble in the culture medium. As measured by the absorbance at 490-500 nm, the amount of formazan produced is directly proportional to the number of living cells. Absorbance was measured using a GloMax plate reader.

FDA-PI (Fluorescein Diacetate-Propidium Iodide) staining live/Dead assay: Dyes solution of FDA (Sigma F7378}: 1:500 dilution and PI (Sigma P4864) 1:150 dilution was added to the samples followed by incubation for 10 min. FDA is a non-fluorescent compound that can freely permeate live cell membranes. Once inside the cells, intracellular esterases cleave the diacetate groups at 37° C., releasing fluorescein, which then emits green fluorescence. Samples were imaged using a light inverted microscope (Olympus IX83, Olympus, Japan) or measured using a GloMax plate reader (Promega).

### Viability and Proliferation Analysis of Chondrocyte Cultures:

Cells were collected from the gelatin ink formulation before and after printing, seeded in triplicate at 3,000 cells/well density in 96-well plates, and incubated in a growth medium. Culture viability was analyzed using RealTime-Glo assay, FDA/PI or Calcein-AM/PI (green, live,/red, dead) staining or counted using the trypan blue exclusion staining. Cells were mixed with Trypan Blue (Biological Industries) at a 1:1 ratio. Live and dead cells were counted using a CellDrop BF Bright Field cell counter (DeNovix) [FIGS. 13-19].

### Histology and Immunostaining

After 7 days of culture, cell-seeded scaffolds were fixed (4% formaldehyde) for 24 hours and embedded in paraffin at 65° C. The scaffolds were sectioned at 5 μm thickness, deparaffinized with OmniPrep solution (ZytoMed) for 40 minutes at 80° C., rehydrated, and stained with hematoxylin and eosin (H&E) for morphological analysis. For immunostaining, sections were permeabilized, washed with PBS, and blocked with BSA solution for 1 hour. Sections were then incubated with a Ki67 primary rabbit monoclonal antibody (1:300, ePredia by Fisher Scientific) according to the manufacturer's instructions, mounted, and imaged using a light inverted microscope (Olympus IX83, Olympus, Japan). Phalloidin-iFluor 647 reagent (Abcam) is used to label F-actin according to the manufacturer's instructions.

### Statistical Analysis

Statistical analysis was performed using a two-tailed unpaired Student's t-test. Differences between groups were considered statistically significant when the p-value was ≤0.05. P-values symbols legend:

| | |
|---|---|
| >0.05 | ns |
| ≤0.05 | * |
| ≤0.001 | ** |
| ≤0.0001 | *** |
| ≤0.00001 | **** |
| ≤0.000001 | ***** |
| ≤0.0000001 | ****** |

1. A 3D printed multilayered biological scaffold, the scaffold comprising a crosslinked native or non-modified protein comprising di-tyrosine bonds.

2. The scaffold according to claim 1, the scaffold containing living cells introduced thereto during printing or post-printing.

3. The scaffold according to claim 1, the scaffold containing materials that enhance cells viability and proliferation; and/or further comprising factors for cartilage and bond tissue engineering.

4. The scaffold according to claim 3, wherein the factors are growth factors, differentiation factors, and/or factors promoting adhesion, proliferation or survival.

5. The scaffold according to claim 2, the scaffold containing materials that enhance cells viability and proliferation; and/or further comprising factors for cartilage and bond tissue engineering.

6. The scaffold according to claim 1, the scaffold being configured for inducing or stimulating tissue growth in vivo, the scaffold comprising printed layers of a crosslinked native or non-modified protein comprising di-tyrosine bonds; and cells capable of inducing or stimulating the growth in vivo.

7. The scaffold according to claim 1, wherein the protein is selected from gelatin, collagen, albumin, silk fibroin and mixtures thereof.

8. The scaffold according to claim 1, formed by vat photopolymerization.

9. The scaffold according to claim 2, wherein the living cells are selected from chondrocytes, osteoblasts, osteocytes, osteoclasts and mixtures thereof.

10. A 3D printed multilayered gelatin scaffold, the scaffold comprising a crosslinked native or non-modified gelatin having a di-tyrosine bonds; the scaffold comprising living cells introduced thereto during printing or post-printing.

11. The scaffold according to claim 10, consisting biological materials.

12. A method of preparing a scaffold according to claim 1, the method comprising visible light-mediated 3D printing of a protein-based formulation to form a 3D multilayered scaffold structure, wherein the protein-based formulation comprises or essentially consists at least one native non-modified protein having an abundance of tyrosine amino acids, a water-soluble photo-initiator, at least one electron acceptor, a liquid carrier and optionally living cells.

13. The method according to claim 12, wherein the formulation is maintained at a temperature not exceeding about 37° C. when cells are used, and not exceeding about 50° C. when cells are not present.

14. The method according to claim 12, wherein a cell-less scaffold is immersed in a formulation comprising the cells; or a cell-less scaffold is treated with a formulation of cells to cause said cells to seed or penetrate the scaffold or associate to the scaffold surface; or a cell-less scaffold is configured to receive thereinto cells migrated subsequent to instillation in the tissue.

15. The method according to claim 12, wherein the protein is gelatin and the formulation comprises cells capable of inducing or stimulating growth of tissue or bone.

16. The method according to claim 12, wherein the photo-initiator is a ruthenium-based photo-initiator, optionally provided with an electron acceptor.

17. The method according to claim 12, the method comprising irradiating by a light having a wavelength in the visible range the aqueous formulation comprising (i) at least one native non-modified protein, being optionally gelatin, having an abundance of tyrosine amino acids, (ii) a water-soluble photo-initiator, (iii) at least one electron acceptor and (iv) living cells, causing the tyrosine amino acids to form di-tyrosine bonds transforming said formulation into said biological scaffold.

18. A method for inducing or stimulating cell or tissue growth or generation in vivo or ex vivo, the method comprising instilling or positioning a biological 3D printed scaffold according to claim 1 in a tissue or an organ, wherein said scaffold optionally comprises cells capable of recruiting host cell infiltration, promotion of tissue growth, and/or tissue regeneration.

19. The method according to claim 18, comprising a step of seeding or treating the scaffold with living cells.

20. The method according to claim 12, wherein the scaffold further comprises active agents that improve the condition of the subject, stimulate healing of an injured tissue or organ, facilitate hastened healing, or reduce pain.
